(19) United States
(12) Patent Application Publication
(10) Pub. No.: US 2023/0320651 A1
(43) Pub. Date: Oct. 12, 2023

(54) BIOLOGICAL SIGNAL MEASUREMENT SYSTEM

(71) Applicants: Shoko TSUCHIMINE, Ishikawa (JP); Hirofumi MORISE, Kanagawa (JP); Kiwamu KUDO, Ishikawa (JP); Yoshihiro MISAKA, Ishikawa (JP); Masato KOIKE, Kagawa (JP)

(72) Inventors: Shoko TSUCHIMINE, Ishikawa (JP); Hirofumi MORISE, Kanagawa (JP); Kiwamu KUDO, Ishikawa (JP); Yoshihiro MISAKA, Ishikawa (JP); Masato KOIKE, Kagawa (JP)

(21) Appl. No.: 18/118,131

(22) Filed: Mar. 7, 2023

(30) Foreign Application Priority Data
Mar. 22, 2022 (JP) 2022-046152

(51) Int. Cl. A61B 5/00

(52) U.S. Cl. CPC A61B 5/4041; A61B 5/7203; A61B 5/7257

(57) ABSTRACT

A biological signal measurement system includes: a providing unit configured to provide a first visual stimulus including a first object visually changing at a predetermined frequency, and a second visual stimulus including a second object; a detection unit configured to detect a biological signal of the subject; a frequency analysis unit configured to perform a frequency analysis on the detected biological signal corresponding to the first visual stimulus, and derive a signal intensity of each frequency component; a determination unit configured to determine a time interval in which the subject has viewed the first visual stimulus, based on a signal intensity of a frequency component corresponding to the frequency; an extraction unit configured to extract the biological signal corresponding to the determined time interval, and corresponding to the second visual stimulus; and an output unit configured to output the extracted biological signal.

### CROSS-REFERENCE TO RELATED APPLICATIONS

The present application claims priority under 35 U.S.C. §119 to Japanese Patent Application No. 2022-046152, filed on Mar. 22, 2022. The contents of which are incorporated herein by reference in their entirety.

### BACKGROUND OF THE INVENTION

### 1. Field of the Invention

The present invention relates to a biological signal measurement system.

### 2. Description of the Related Art

Conventionally, a visual stimulus is given to a subject and a biological reaction against the visual stimulus is measured. For example, with use of a brain function measurement apparatus, such as a magnetoencephalography, a brain reaction against a given visual stimulus is measured as a biological reaction.

Meanwhile, to capture the biological reaction against the visual stimulus, the subject needs to continue to view an image that gives the visual stimulus. In this case, with an increase in a measurement time, concentration of the subject decreases, so that a ratio of a biological reaction that is irrelevant to the reaction against the visual stimulus increases and it becomes difficult to capture the biological reaction with high accuracy. To cope with this, conventionally, a technology of embedding frequency information in images that give visual stimuli and distinguishing a type of an image that is viewed by the subject has been proposed (for example, Japanese Unexamined Patent Application Publication No. 2013-4006).

However, in the conventional technology, a biological reaction during a period in which the subject does not view a stimulus image is also acquired as a measurement result, so that accuracy of the acquired data may be reduced.

### SUMMARY OF THE INVENTION

According to one aspect of the present invention, a biological signal measurement system includes a providing unit, a detection unit, a frequency analysis unit, a determination unit, an extraction unit, and an output unit. The providing unit is configured to provide a first visual stimulus and a second visual stimulus to a subject. The first visual stimulus includes a first object visually changing at a predetermined frequency. The second visual stimulus includes a second object. The detection unit is configured to detect a biological signal of the subject. The frequency analysis unit is configured to perform a frequency analysis on the biological signal detected by the detection unit and corresponding to the first visual stimulus, and derive a signal intensity of each frequency component. The determination unit is configured to determine a time interval in which the subject has viewed the first visual stimulus, based on a signal intensity of a frequency component corresponding to the frequency. The extraction unit is configured to extract the biological signal corresponding to the time interval determined by the determination unit, and corresponding to the second visual stimulus. The output unit is configured to output the biological signal extracted by the extraction unit.

### BRIEF DESCRIPTION OF THE DRAWINGS

FIG. 1 is a diagram illustrating an example of a system configuration of a biological information measurement system according to one embodiment;

FIGS. 2A and 2B are diagrams illustrating an example of a visual stimulus that is provided by a visual stimulus providing apparatus according to the embodiment;

FIGS. 3A and 3B are diagrams illustrating an example of a change in luminance of a first object according to the embodiment;

FIG. 4 is a diagram illustrating an example of a visual change of the first object according to the embodiment;

FIG. 5 is a diagram illustrating an example of a hardware configuration of a biological signal detection apparatus according to the embodiment;

FIG. 6 is a diagram illustrating an example of a functional configuration of the biological signal detection apparatus according to the embodiment;

FIG. 7 is a diagram illustrating an example of biological signals that are detected by a sensor unit of the embodiment;

FIG. 8 is a diagram illustrating an example of biological signals that are processed by an analysis unit of the embodiment;

FIG. 9 is a diagram illustrating another example of the biological signal that is processed by the analysis unit of the embodiment;

FIG. 10 is a diagram illustrating an example of biological information that is extracted by an extraction unit of the embodiment;

FIG. 11 is a flowchart illustrating an example of a process performed by the biological signal detection apparatus of the embodiment;

FIG. 12 is a diagram for explaining an example of a screen for a visual stimulus according to a first modification;

FIG. 13 is a diagram for explaining an example of a screen for a visual stimulus according to a second modification; and

FIG. 14 is a diagram for explaining an example of a screen for a visual stimulus according to a third modification.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

Embodiments of a biological signal measurement system will be described in detail below with reference to the accompanying drawings.

An embodiment has an object to measure a biological reaction of a subject against a visual stimulus with high accuracy.

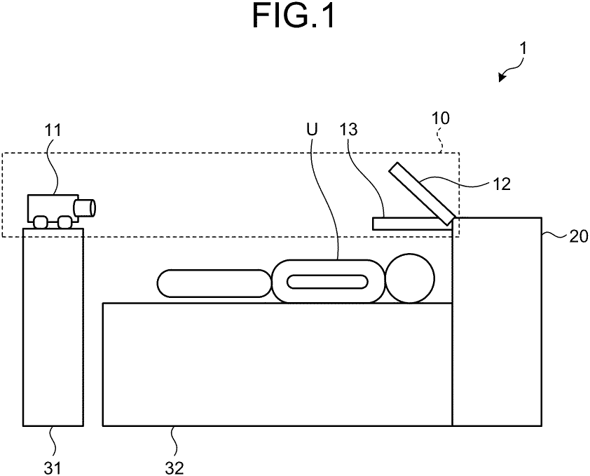

FIG. 1 is a diagram illustrating an example of a system configuration of a biological signal measurement system 1 according to one embodiment. As illustrated in FIG. 1, a biological signal measurement system 1 includes a visual stimulus providing apparatus 10 and a biological signal measurement apparatus 20.

The visual stimulus providing apparatus 10 is one example of a providing unit. The visual stimulus providing apparatus 10 provides an image (including a still image and a moving image, and hereinafter, also referred to as a visual stimulus) that gives a visual stimulus to a subject U. For example, as illustrated in FIG. 1, the visual stimulus providing apparatus 10 includes an image projection unit 11, a mirror 12, and a screen 13.

The image projection unit 11 is a projection device, such as a projector, that projects image light. The image projection unit 11 is mounted on a mounting table 31 or the like and projects image light toward the mirror 12 that is arranged above the subject U who lies on his/her back on a bed 32. The mirror 12 and the screen 13 are arranged in front of eyes of the subject U who lies on his/her back on the bed 32. The mirror 12 reflects the image light that is projected by the image projection unit 11, and projects the image light on the screen 13.

In the configuration as described above, the visual stimulus providing apparatus 10 projects a visual stimulus from the image projection unit 11, and provides the visual stimulus in front of the eyes of the subject U.

Meanwhile, in the present embodiment, the visual stimulus providing apparatus 10 is configured to project a visual stimulus on the screen 13, but embodiments are not limited to this example. For example, the visual stimulus providing apparatus 10 may display a visual stimulus on a display device, such as a liquid crystal display (LCD). Further, for example, the visual stimulus providing apparatus 10 may display a visual stimulus by virtual reality (VR) or the like on a head-mounted display or the like.

The biological signal measurement apparatus 20 includes one or more sensor units 27 (see FIG. 5), such as potential sensors or magnetic sensors, and acquires, as a biological signal, a potential or a magnetic field that is generated by a living body of the subject U via the sensor units 27. Further, the biological signal measurement apparatus 20 analyzes the biological signal that is detected from the subject U, and outputs an analysis result. An output destination of the analysis result is not specifically limited, and the analysis result may be output to a display device or a printing device (not illustrated), or data of the analysis result may be output to a storage device or a different device.

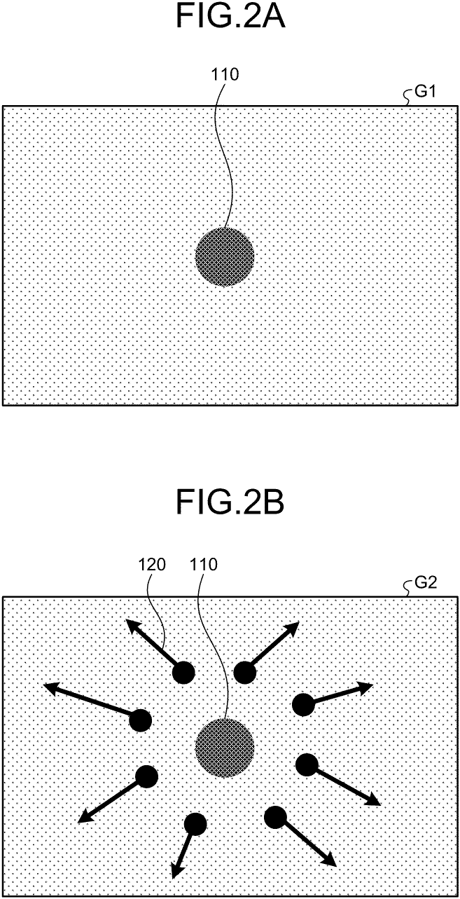

The visual stimulus that is provided by the visual stimulus providing apparatus 10 will be described below with reference to FIG. 2A to FIG. 4. Meanwhile, provision (display) of the visual stimulus by the visual stimulus providing apparatus 10 may be controlled by an external apparatus other than the apparatuses illustrated in the biological signal measurement system 1; however, in the present embodiment, explanation will be given based on the assumption that the biological signal measurement apparatus 20 controls the provision (display).

FIGS. 2A and 2B are diagrams illustrating an example of the visual stimulus that is provided by the visual stimulus providing apparatus 10. Here, FIG. 2A illustrates a first visual stimulus G1 that is provided by the visual stimulus providing apparatus 10. Further, FIG. 2B illustrates a second visual stimulus G2 that is provided by the visual stimulus providing apparatus 10.

As illustrated in FIG. 2A and2B, each of the first visual stimulus G1 and the second visual stimulus G2 includes a first object 110. The first object 110 is fixedly arranged in a screen, and is formed in a certain shape, such as a circular shape, a polygonal shape, a cross shape, or a combination of a circular shape, a polygonal shape, a cross shape, and the like. Meanwhile, an inside of the first object 110 may be uniformly painted, or a pattern, a photograph, an illustration, a character, or the like may be drawn inside the first object 110.

The first object 110 is arranged in, for example, approximately the center of the screen of the visual stimulus. As one example, at least a part of the first object 110 is arranged in a central section when the entire screen is divided into nine sections such that each of a vertical side and a horizontal size is equally divided into three sections. Meanwhile, it is preferable that the first object 110 is arranged in a range in which a viewing angle of the subject U with respect to the center of the screen is equal to or smaller than 10 degrees.

Further, a size of the first object 110 is set to 1% or more of a size of the screen or such that a visual field from the subject U is equal to or larger than 3 to 10 degrees. By providing the first object 110 that is arranged as described above, the subject U is able to easily view the first object 110, so that it is possible to prevent the first object 110 from being excluded from a viewing target.

Furthermore, in all or a part of a region of the first object 110, luminance that represents the region changes at a predetermined frequency (hereinafter, also referred to as a change frequency fl) of 10 Hz or more. Meanwhile, it is preferable to set the change frequency f1 to 60 Hz or more to ensure safety, such as prevention of possibility of occurrence of photosensitive epilepsy in the subject. Moreover, it is preferable to set an upper limit to about 200 Hz because a higher frequency is less likely to be detected as a brain reaction. Furthermore, to obtain an adequate brain reaction, it is preferable to change a region that occupies at least 30% or more of an area of the first object 110. In the following, a visual change of a part or all of the first object 110 at the change frequency f1 may also be referred to as embedding of frequency information in the first object 110.

Meanwhile, a method of changing the luminance of the first object 110 is not specifically limited, and, for example, it may be possible to change the luminance in a continuous manner or in a stepped manner or it may be possible to switch between two states such as a bright state and a dark state in a cyclic manner.

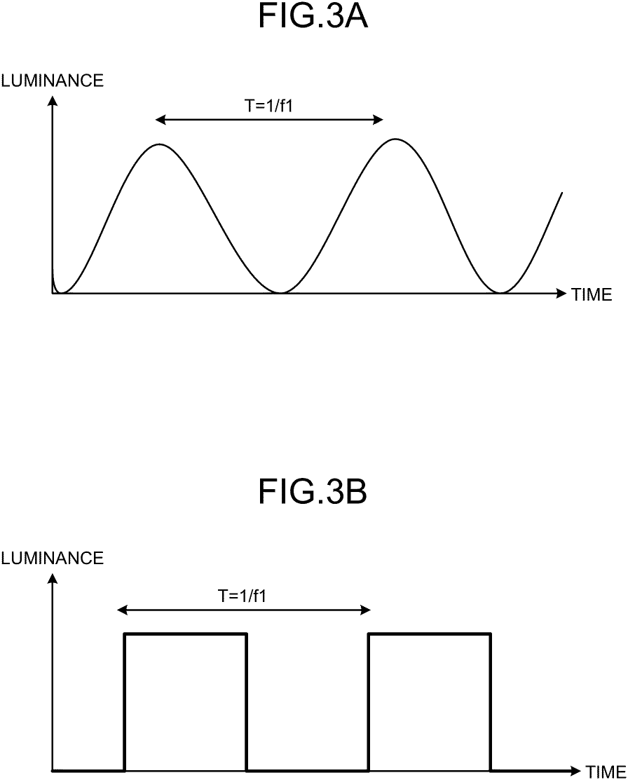

FIGS. 3A and 3B are diagrams illustrating a change in the luminance of the first object 110. Here, a horizontal axis represents an elapsed time and a vertical axis represents the luminance. Further, T illustrated in the figure represents a cycle (⅟f1) of the change frequency f1.

Here, FIG. 3A illustrates an example in which the luminance of the first object 110 is changed based on a sine wave. In this case, the luminance continuously changes between the two states such as the bright state and the dark state. In contrast, FIG. 3B illustrates an example in which the luminance of the first object 110 is changed based on a square wave. In this case, the luminance is changed between the two states such as the bright state and the dark state for each T/2. Meanwhile, in the case illustrated in FIG. 3B, the luminance need not always be changed between the two states such as the bright state and the dark state, but may be changed among three or more states.

The luminance in the first object 110 may be uniformly changed or may be changed with a certain spatial pattern. Further, it is preferable that a region in which the luminance changes has a pattern, such as a stripe, a dot, a ring, or a lattice, that is formed of a plurality of constituent elements because it becomes easy to recognize a difference in a stimulus frequency.

Furthermore, the visual change of the first object 110 may be realized using other than the luminance. For example, it may be possible to change a color, a pattern, or the like that represents all or a part of the first object 110 based on the change frequency f1.

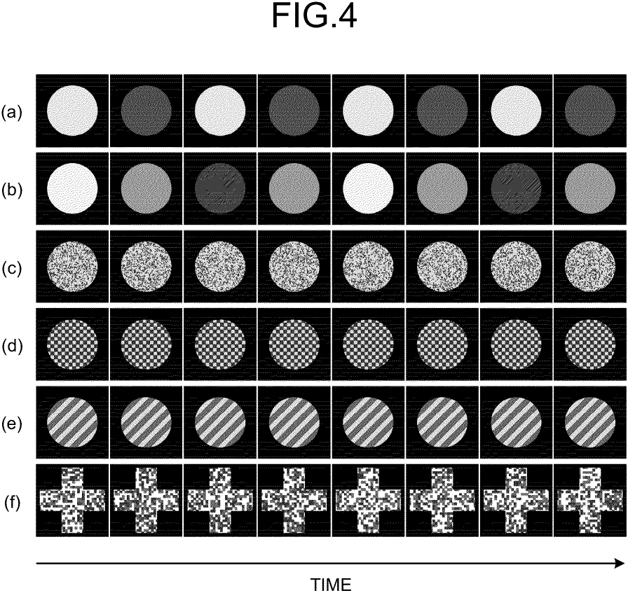

FIG. 4 is a diagram illustrating a visual change of the first object 110. In FIG. 4, a horizontal direction represents an elapsed time at a certain time interval. Further, examples of various visual changes are arranged in a vertical direction.

For example, FIG. 4 illustrates, at (a), an example in which the luminance of the circular first object 110 is changed using binary values. FIG. 4 illustrates, at (b), an example in which the luminance of the circular first object 110 is changed using three or more values (including a continuous change based on a sine wave). FIG. 4 illustrates, at (c), an example in which a dot noise pattern is drawn in the circular first object 110 and the pattern is changed. FIG. 4 illustrates, at (d), an example in which a lattice pattern is drawn in the circular first object 110 and the pattern is changed by being reversed. FIG. 4 illustrates, at (e), an example in which a stripe pattern is drawn in the circular first object 110 and the pattern is changed by being reversed or moved. FIG. 4, at (f), illustrates an example in which a dot noise pattern is drawn in the cross-shaped first object 110 and the pattern is changed.

Furthermore, the second visual stimulus G2 illustrated in FIG. 2B includes second objects 120. Each of the second objects 120 is formed in a circular shape, a polygonal shape, a dot shape, or a combination of the circular shape, the polygonal shape, the dot shape, and the like. Moreover, an inside of each of the second objects 120 may be uniformly painted, or a pattern, a photograph, an illustration, a character, or the like may be drawn inside each of the second objects 120.

Furthermore, each of the second objects 120 may be a visual stimulus for detecting a brain reaction or a visual stimulus for measuring, for example, a cognitive function, such as a spatial cognitive function, a language cognitive function, an executive function, a working memory, or an attention, phonological processing, or a character morphology processing.

A state of each of the second objects 120 temporally changes by a predetermined program. For example, display and non-display may be switched at a predetermined time interval in a range from 50 milliseconds to 10 seconds. Further, each of the second objects 120 may be provided with motion, such as enlargement, reduction, movement, or rotation, when being displayed. Meanwhile, in FIG. 2B, the example is illustrated in which the plurality of second objects 120 that are arranged around the first object 110 are moved in a radial manner.

In the present embodiment, the first visual stimulus G1 and the second visual stimulus G2 as described above are provided as a moving image (video) in a continuous manner. For example, the biological signal measurement apparatus 20 first causes the visual stimulus providing apparatus 10 to provide the first object 110 that is arranged at a fixed point and that flickers at the change frequency f1, and thereafter, causes the visual stimulus providing apparatus 10 to provide the second objects 120 that are provided with motion, such as enlargement, reduction, movement, or rotation, while continuously providing the first object 110. Further, the first object 110 and the second objects 120 are repeatedly provided.

Meanwhile, if the first visual stimulus G1 and the second visual stimulus G2 are still images, it may be possible to first provide the first visual stimulus G1, and thereafter provide the second visual stimulus G2 in a switched manner. Furthermore, a pattern of providing the visual stimulus is not limited to the example as described above.

Hereinafter, operation from start of provision of the first object 110 (the first visual stimulus G1) to completion of provision of the second objects 120 (the second visual stimulus G2) may also be referred to as a single trial. In other words, the biological signal measurement apparatus 20 causes the visual stimulus providing apparatus 10 to repeatedly provide visual stimuli by adopting a series of visual stimuli such as the first visual stimulus G1 and the second visual stimulus G2 as a single trial. The single trial takes about 3 seconds, for example.

The biological signal measurement apparatus 20 will be described below. FIG. 5 is a diagram illustrating an example of a hardware configuration of the biological signal measurement apparatus 20. As illustrated in FIG. 5, the biological signal measurement apparatus 20 includes a central processing unit (CPU) 21, a read only memory (ROM) 22, a random access memory (RAM) 23, a storage unit 24, a display unit 25, an operating unit 26, the sensor units 27, and an interface unit 28.

The CPU 21 is one example of a processor and integrally controls each of the units of the biological signal measurement apparatus 20. The ROM 22 stores therein various programs. The RAM 23 is a workspace for loading a program and various kinds of data. The CPU 21, the ROM 22, and the RAM 23 realize a computer configuration of the biological signal measurement apparatus 20, and function as a control unit of the biological signal measurement apparatus 20.

The storage unit 24 is a storage device, such as a hard disk drive (HDD), a flash memory, or the like. The storage unit 24 stores therein various programs that are executed by the CPU 21, setting information, or the like. Further, the storage unit 24 stores therein data of a visual stimulus that is provided by the visual stimulus providing apparatus 10. Furthermore, the storage unit 24 also functions as a storage region for storing a biological signal that is detected from the subject U.

The display unit 25 is a display device, such as a liquid crystal display (LCD). The display unit 25 displays various kinds of information and a screen under the control of the CPU 21. The operating unit 26 includes an input device, such as a keyboard or a mouse, and outputs a signal corresponding to user operation to the CPU 21. Meanwhile, the operating unit 26 may be a touch panel that is arranged on a surface of the display unit 25.

The sensor unit 27 is one example of a detection unit. The sensor unit 27 is a sensor device, such as a potential sensor or a magnetic sensor, and detects a potential or a magnetic field that is generated by a living body of the subject U as a biological signal. Specifically, the sensor unit 27 repeatedly measures, as the biological signal, a change of a potential, a magnetic field, or the like that is generated by a pulse, blood pressure, a respiratory rate, or neuro-electric activity of the subject U. Meanwhile, in the present embodiment, an example will be described in which brain neural activity of the subject U is detected as the biological signal, but a detection target is not specifically limited.

The interface unit 28 is a wired or wireless interface for connecting the visual stimulus providing apparatus 10 or the like.

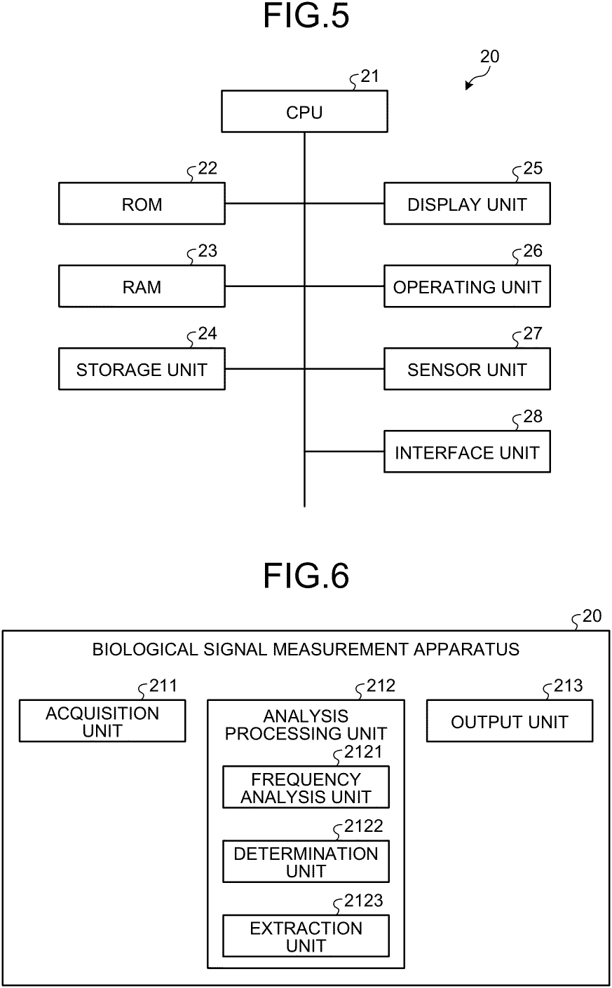

A functional configuration of the biological signal measurement apparatus 20 will be described below. FIG. 6 is a diagram illustrating an example of the functional configuration of the biological signal measurement apparatus 20. As illustrated in FIG. 6, the biological signal measurement apparatus 20 includes, as functional components, an acquisition unit 211, an analysis processing unit 212, and an output unit 213.

A part or all of the functional components included in the biological signal measurement apparatus 20 may be software components that are implemented by cooperation of a processor (for example, the CPU 21) of the biological signal measurement apparatus 20 and a program that is stored in the memory (for example, the ROM 22 or the storage unit 24). Further, a part or all of the functional components included in the biological signal measurement apparatus 20 may be hardware components that are implemented by a dedicated circuit or the like that is mounted on the biological signal measurement apparatus 20.

The acquisition unit 211 acquires, in cooperation with the sensor unit 27, the biological signal of the subject U that is detected by the sensor unit 27. Specifically, the acquisition unit 211 acquires, as the biological information, a data group of biological signals that are repeatedly measured for each of trials by the sensor unit 27.

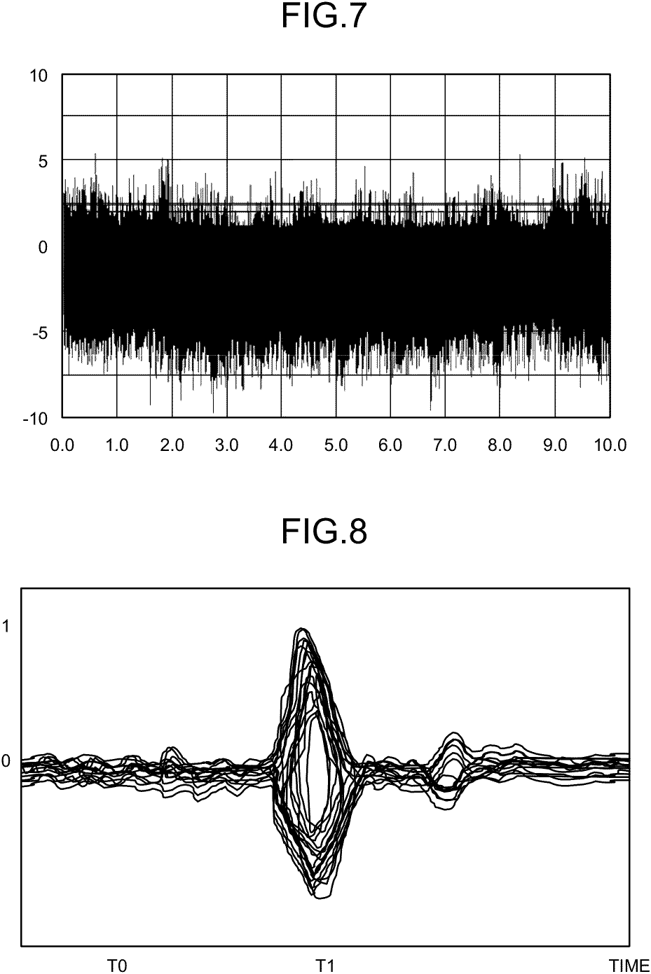

FIG. 7 is a diagram illustrating an example of the biological signals that are detected by the sensor unit 27. Here, FIG. 7 illustrates all of biological signals detected by the sensor unit 27. The biological signals include various kinds of noise. Meanwhile, a horizontal axis represents a time axis and a vertical axis represents signal intensity.

As illustrated in FIG. 7, the biological signals that are detected by the sensor unit 27 are obtained as waveform data. The acquisition unit 211 acquires, as biological information, a data group of the biological signals that are repeatedly measured by the sensor unit 27 for each of conditions (for example, types of visual stimuli).

Further, the acquisition unit 211 inputs the acquired biological information to the analysis processing unit 212 in addition to a measurement condition that is adopted when the biological information is measured. Examples of the measurement condition include a measurement time, an attribute of the subject U, and a type of the visual stimulus. For example, the acquisition unit 211 acquires a measurement time at which each of the trials is performed, a type of the visual stimulus, or the like, in cooperation with the sensor unit 27, a time measurement unit (not illustrated), such as a real time clock (RTC), or the like of the visual stimulus providing apparatus 10. Furthermore, for example, the acquisition unit 211 acquires the attribute (age, gender, or the like) of the subject U that is input via the operating unit 26 or the like.

Meanwhile, the acquisition unit 211 may input the acquired biological information and the acquired measurement condition in real time to the analysis processing unit 212, or may store the acquired biological information and the acquired measurement condition in the storage unit 24 or the like and thereafter input the biological information and the measurement condition that are obtained through a series of the trials to the analysis processing unit 212.

Moreover, the acquisition unit 211 may calculate a statistical value (an average value, a maximum value, a minimum value, a median value, a dispersion, or the like) from the biological information that is acquired in a predetermined time rather than inputting the biological information as it is to the analysis processing unit 212, and input the calculated statistical value to the analysis processing unit 212.

The analysis processing unit 212 analyzes the biological information (biological signal) that is detected by the sensor unit 27, and outputs the biological information that is obtained when the subject U views the visual stimulus. The analysis processing unit 212 adopts the biological information (for example, magnetic field data) on the brain activity that is input from the acquisition unit 211 as an analysis target. Meanwhile, the analysis target is not limited to the brain activity, but may be a pulse, blood pressure, a respiratory rate, a cerebral blood flow, eye motion, body motion, or the like of the subject U.

Specifically, the analysis processing unit 212 includes a frequency analysis unit 2121, a determination unit 2122, and an extraction unit 2123.

The frequency analysis unit 2121 performs a frequency analysis on the biological information, and derives signal intensity for each frequency component. Specifically, the frequency analysis unit 2121 performs short-time Fourier transform on a biological signal (biological information) that is detected in a time interval [T1-Δt, T1+Δt] around a time T1.

Here, for example, an intermediate time or the like of each of the trials is set as the time T1, and Δt is set to a value such that the time interval [T1-Δt, T1+Δt] is equal to or smaller than a duration of a single trial. In the present embodiment, explanation will be given based on the assumption that the time interval [T1-Δt, T1+Δt] corresponds to a duration of each of the trials. Further, the time interval [T1-Δt, T1+Δt] is included in a time in which the first object 110 (or the first visual stimulus G1) is being provided.

Furthermore, it is preferable that the frequency analysis unit 2121 performs the frequency analysis using biological information corresponding to at least two trials. More specifically, the frequency analysis unit 2121 performs short-time Fourier transform using the biological information corresponding to the trials, and derives, as a frequency analysis result, a result of an arithmetic mean of the waveforms that are subjected to the short-time Fourier transform. With this configuration, it is possible to reduce an influence of noise, so that it is possible to improve accuracy of the biological signal.

Moreover, the frequency analysis unit 2121 may perform a filtering process using a frequency response filter (for example, a band-pass filter, a low-pass filter, a high-pass filter, or a notch filter) before or after the frequency analysis as described above, and perform a process of eliminating noise in a certain frequency band that is different from the frequency band of the biological signal.

Furthermore, the frequency analysis unit 2121 may eliminate, as noise, information, such as a heart rate, myoelectric, or blink, that is other than the brain activity and that is included in the biological signal, using a principal component analysis (PCA), an independent component analysis (ICA), or the like in order to narrow down the information on the brain activity.

FIG. 8 is a diagram illustrating an example of biological signals that are subjected to the frequency analysis by the frequency analysis unit 2121. A horizontal axis represents a time axis and a vertical axis represents signal intensity of the biological signals (magnetic data) related to the brain activity. Further, the waveforms in FIG. 8 are arithmetic means of the biological signals that are subjected to the frequency analysis. Meanwhile, in FIG. 8, T0 corresponds to, for example, T1-Δt.

As illustrated in FIG. 8, the biological signals that are processed by the frequency analysis unit 2121 are signals from which various noises are eliminated, and represent the brain activity of the subject U. In FIG. 8, peaks of the biological signals appear at a time T1. Here, the time T1 corresponds to a timing at which a visual stimulus (for example, the second objects 120) to be measured is provided.

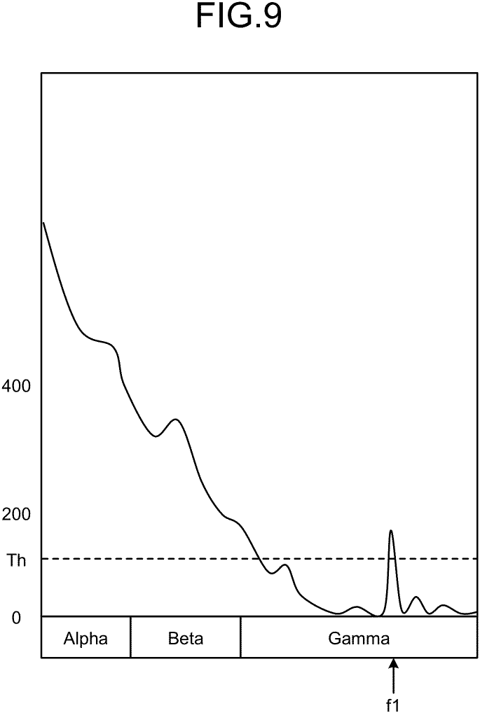

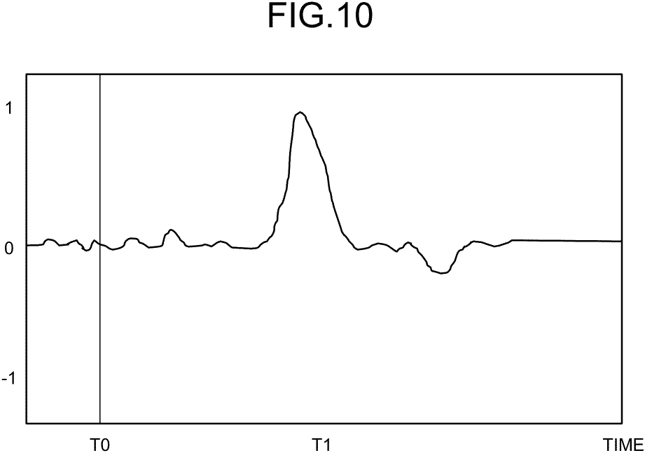

Referring back to FIG. 6, the determination unit 2122 determines the time interval [T1-Δt, T1+Δt] in which the subject U views the visual stimulus, based on signal intensity of a frequency component that corresponds to the change frequency f1 or a multiple of the change frequency f1 among the frequency components of the biological information that is processed by the frequency analysis unit 2121. Operation of the determination unit 2122 will be described below with reference to FIG. 9.

FIG. 9 is a diagram illustrating an example of the biological information that is processed by the frequency analysis unit 2121. In FIG. 9, the biological information is represented by signal intensity (power spectrum) of each frequency component. A horizontal axis represents a frequency, and “Alpha”, “Beta”, and “Gamma” correspond to frequency bands of an alpha wave, a beta wave, and a gamma wave, respectively. Further, a vertical axis represents the signal intensity.

When the subject U views the visual stimulus, the subject U receives the visual stimulus of the first object 110, so that a brain reaction against the change frequency f1 appears in the biological information. For example, in FIG. 9, a waveform of the signal intensity corresponding to the change frequency f1 corresponds to the brain reaction, that is, the biological reaction against the visual stimulus of the first object 110.

Therefore, as illustrated in FIG. 9, the determination unit 2122 compares the signal intensity of the frequency component corresponding to the change frequency f1 and a threshold Th that is determined in advance. Then, if the signal intensity exceeds the threshold Th, the determination unit 2122 determines that the subject U has viewed the visual stimulus during the time interval [T1-Δt, T1+Δt] corresponding to the biological information. In other words, the determination unit 2122 identifies the time interval [T1-Δt, T1+Δt] during which the subject U has viewed the visual stimulus.

In contrast, if the signal intensity of the frequency component corresponding to the change frequency f1 is equal to or smaller than the threshold Th, the determination unit 2122 determines that the subject U has not viewed the visual stimulus during the time interval [T1-Δt, T1+Δt] corresponding to the biological information.

Meanwhile, it is possible to set the threshold Th to an arbitrary value, but it is preferable to set the threshold Th to a value by which it is possible to significantly identify whether the visual stimulus has been viewed. For example, it may be possible to determine that the visual stimulus has been viewed if an S/N ratio of the signal intensity of the frequency component corresponding to the change frequency f1 is about two times or more. Further, it is preferable that the change frequency f1 includes a plurality of frequencies because it becomes possible to improve determination accuracy, and, in this case, it may be possible to set a threshold for each frequency.

The extraction unit 2123 selects biological information based on the determination result of the determination unit 2122. Specifically, the extraction unit 2123 extracts the biological information corresponding to the time interval [T1-Δt, T1+Δt] that is determined by the determination unit 2122 as a time interval in which the visual stimulus has been viewed among the pieces of biological information processed by the frequency analysis unit 2121. Furthermore, the extraction unit 2123 discards the biological information corresponding to the time interval [T1-Δt, T1+Δt] that is determined by the determination unit 2122 as a time interval in which the visual stimulus has not been viewed, and excludes the biological information from an extraction target.

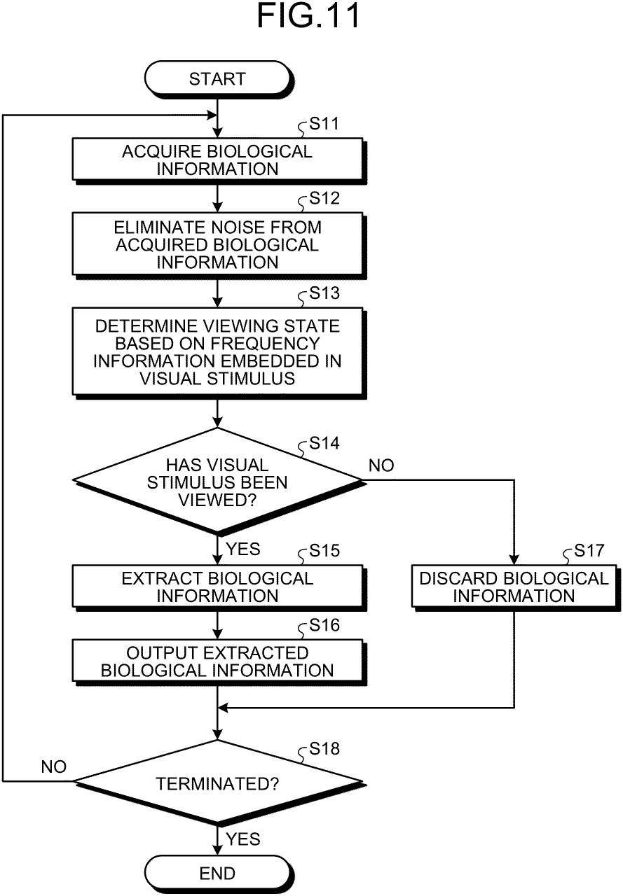

FIG. 10 is a diagram illustrating an example of the biological information that is extracted by the extraction unit 2123. Meanwhile, FIG. 10 illustrates a result of extraction of the biological information corresponding to the time interval [T1-Δt, T1+Δt] in which the subject U has viewed the visual stimulus from among the pieces of biological information for which the arithmetic means are obtained in FIG. 8. Meanwhile, T0 corresponds to, for example, T1-Δt.

As illustrated in FIG. 10, the extraction unit 2123 extracts the biological information corresponding to the time interval [T1-Δt, T1+Δt] in which the subject U has viewed the visual stimulus. With this configuration, the biological signal measurement apparatus 20 is able to measure the brain reaction when the subject U views the visual stimulus with high accuracy.

Referring back to FIG. 6, the output unit 213 outputs the biological information that is extracted by the analysis processing unit 212 (the extraction unit 2123). Specifically, the output unit 213 outputs a result of an arithmetic mean of the biological information (biological signals) extracted by the extraction unit. With this configuration, the output unit 213 is able to obtain an arithmetic mean of only the biological signals that represent the brain reaction that is caused by viewing of the visual stimulus, so that it is possible to improve measurement accuracy of the brain reaction.

Meanwhile, an output method of the output unit 213 is not specifically limited. For example, the output unit 213 may be configured to store the biological information and the measurement condition by outputting the biological information and the measurement condition to the storage unit 24. Further, for example, the output unit 213 may display the biological information and the measurement condition on the display unit 25. Furthermore, for example, the output unit 213 may output the biological information and the measurement condition to an external apparatus via the interface unit 28 or the like.

An example of operation of the biological signal measurement apparatus 20 will be described below with reference to FIG. 11. FIG. 11 is a flowchart illustrating an example of a process performed by the biological signal measurement apparatus 20. Meanwhile, it is assumed that, as a premise of the process, the visual stimulus providing apparatus 10 repeatedly provides the visual stimuli (the first object 110 and the second objects 120) at predetermined time intervals.

First, the acquisition unit 211 acquires the biological information corresponding to one or more trials that are detected by the sensor unit 27 (Step S11). Subsequently, the frequency analysis unit 2121 eliminates noise by performing a frequency analysis on the biological information acquired by the sensor unit 27 (Step S12).

Subsequently, the determination unit 2122 determines a viewing state of the visual stimulus based on the signal intensity of the frequency component corresponding to the frequency information that is embedded in the visual stimulus among the frequency components of the biological information processed at Step S12 (Step S13). Here, if the determination unit 2122 determines that the visual stimulus has been viewed (Step S14; Yes), the extraction unit 2123 extracts the biological information on the corresponding trial (Step S15). Subsequently, the output unit 213 outputs the biological information extracted at Step S15 and the measurement condition in an associated manner (Step S16), and the process goes to Step S18.

In contrast, if the determination unit 2122 determines that the visual stimulus has not been viewed (Step S14; No), the extraction unit 2123 discards the biological information on the corresponding trial (Step S17), and the process goes to Step S18.

At Step S18, the analysis processing unit 212 determines whether an instruction on termination of the process is issued (Step S18). If the instruction on the termination of the process is not issued (Step S18; No) the analysis processing unit 212 returns the process to Step S11.

Furthermore, for example, if the instruction on the termination of the process is issued by the operating unit 26, or if provision of the visual stimulus or sensing operation of the sensor unit 27 is stopped, the analysis processing unit 212 determines that the instruction on the termination of the process is issued (Step S18; Yes), and the process is terminated.

As described above, the biological signal measurement system 1 according to the present embodiment includes the visual stimulus providing apparatus 10 that provides the visual stimulus including the first object 110 that visually changes at the change frequency f1 to the subject U, and the biological signal measurement apparatus 20 that detects biological signals of the subject U. Furthermore, the biological signal measurement apparatus 20 performs a frequency analysis on the detected biological signals, derives signal intensity of each frequency component, determines a time interval in which the subject U has viewed the visual stimulus based on the signal intensity of the frequency component corresponding to the change frequency f1, and extracts and outputs a biological signal corresponding to the time interval. With this configuration, the biological signal measurement system 1 is able to obtain the brain reaction corresponding to a period in which the subject U has viewed the visual stimulus, so that it is possible to measure the brain reaction of the subject U against the visual stimulus with high accuracy.

Meanwhile, the embodiment as described above may be embodied with an appropriate modification by changing a part of the configuration or the function of each of the apparatuses as described above. Therefore, some modifications of the embodiment as described above will be described as different embodiments. Meanwhile, in the following, a difference from the embodiment as described above will be mainly described, and detailed explanation of the same points as the details that are already explained will be omitted. Furthermore, the modifications described below may be implemented individually or by an appropriate combination.

### First Modification

In the embodiment as described above, the example has been described in which the first object 110 is arranged in an approximately center of the screen of the visual stimulus, but the arrangement position of the first object 110 is not limited to this example. For example, as illustrated in FIG. 12, it may be possible to arrange the plurality of first objects 110 in the scree of the visual stimulus.

FIG. 12 is a diagram for explaining an example of a screen of a visual stimulus according to a first modification. As illustrated in FIG. 12, in a visual stimulus G3, at least the single first object 110 is arranged in each quadrant (region) of a screen that is divided into four quadrants.

By providing the visual stimulus G3, the biological signal measurement apparatus 20 is able to effectively obtain the brain reaction because the first object 110 is viewed even if the subject U views any part of the screen. With this configuration, in the biological signal measurement system 1 according to the present modification, it is possible to reduce the number of pieces of biological information to be excluded, that is, the number of trials, so that it is possible to reduce the entire measurement time and reduce a load on the subject U.

### Second Modification

In the embodiment as described above, the example has been described in which the first object 110 and the second objects 120 are provided as different stimulus images or stimulus videos. However, the method of providing the first object 110 and the second objects 120 is not limited to this example. Therefore, in the present modification, as one example of the providing method, a visual stimulus that is used when the first object 110 and the second objects 120 are simultaneously provided will be described.

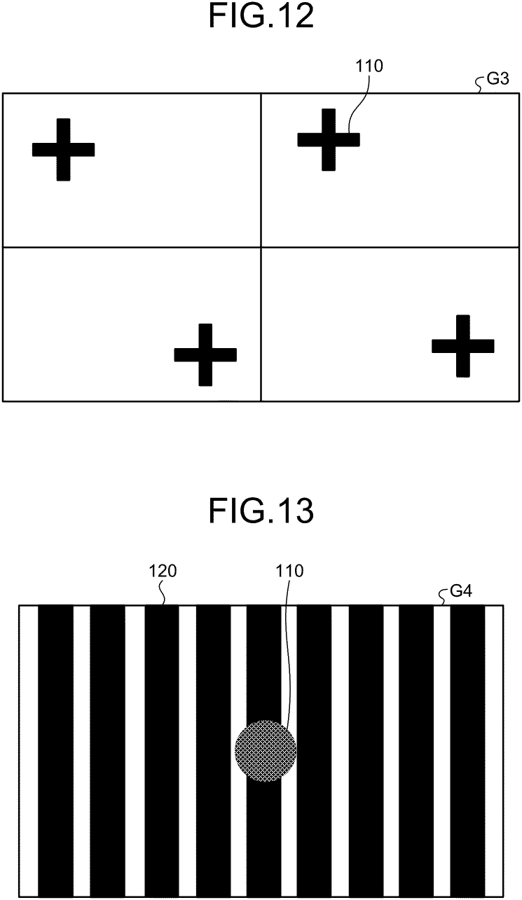

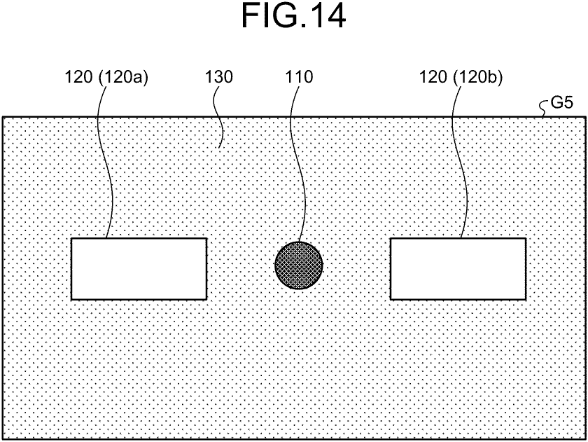

FIG. 13 is a diagram for explaining an example of a screen of a visual stimulus according to a second modification. As illustrated in FIG. 13, a visual stimulus G4 includes the first object 110 that is arranged in approximately the center of the screen and the second objects 120 that are arranged around the first object 110.

Here, the first object 110 is a fixed point and flickers at the change frequency f1. The second objects 120 are represented as a stripe pattern and colored such that white and black alternately appear. Further, the second objects 120 provide a reversal stimulus such that coloring of white and black changes by reversing (or moving) the coloring of white and black.

By providing the visual stimulus G4, the biological signal measurement apparatus 20 is able to simultaneously determine whether the subject U has viewed the first object 110 and acquire the brain reaction generated by the viewing of the second objects 120. Therefore, the biological signal measurement system 1 according to the present modification is able to reduce the trial time, so that it is possible to reduce a load on the subject U.

Meanwhile, the pattern of the second objects 120 is not limited to a stripe pattern, but may be a lattice pattern, a checkered pattern, a concentric circle pattern, or the like.

### Third Modification

In the embodiment as described above, it is explained that the frequency information is embedded in the first object 110. However, the frequency information need not always be embedded in the first object 110.

For example, it may be possible to embed the frequency information in the second objects 120. In this case, it is preferable that a frequency band of the frequency information that is embedded in the first object 110, that is, the change frequency f1 that is used to determine whether the stimulus is viewed, and a frequency band of the frequency information that is embedded in the second objects 120 do not overlap with each other.

With this configuration, it is possible to realize determination on whether the first object 110 is viewed and acquisition of the brain reaction by the second objects 120 by providing a single visual stimulus.

Furthermore, it may be possible to embed the frequency information in a region other than the first object 110 and the second objects 120. FIG. 14 is a diagram for explaining an example of a screen of a visual stimulus according to a third modification, and illustrates an example in which the frequency information is embedded in an entire periphery other than the first object 110 and the second objects 120.

Specifically, a visual stimulus G5 illustrated in FIG. 14 includes the first object 110 that is a fixed point and two second objects 120a and 120b that are rectangular shapes. Here, the frequency information on the change frequency f1 (Hz) is embedded in the first object 110, and the first object 110 flickers at, for example, the change frequency f1.Further, frequency information on a frequency f2 (Hz) is embedded in the second object 120a, and the second object 120a flickers at, for example, the frequency f2. Furthermore, frequency information on a frequency f3 (Hz) is embedded in the second object 120b, and the second object 120b flickers at, for example, a frequency f3. Here, each of the frequencies f1, f2, and f3 belongs to a different frequency band. Meanwhile, in this case, it is preferable that the frequencies f2 and f3 are frequencies of interest that are targets for analysis of the brain reaction.

Furthermore, frequency information on a frequency f4 (Hz) is embedded in a region 130 that is a background of the first object 110 and the second objects 120a and 120b, and the region 130 flickers at, for example, the frequency f4. Meanwhile, a frequency band of the frequency f4 is different from the frequency band of any of the frequencies f1, f2, and f3.

By providing the visual stimulus G5, the biological signal measurement apparatus 20 is able to distinguish one of the second objects 120a and 120b viewed by the subject U. Further, the biological signal measurement apparatus 20 is able to exclude a trial that is performed when the subject U does not view any of the second objects 120a and 120b, such as when the subject U is sleeping. With this configuration, the biological signal measurement system 1 according to the present modification is able to effectively acquire the brain reaction when the second objects 120a and 120b are viewed, so that it is possible to improve measurement accuracy.

Meanwhile, the second objects 120 (120a and 120b) have rectangular shapes in FIG. 14, but are not limited to this example, and may have different shapes, such as circular shapes. Furthermore, the two second objects 120 are provided in FIG. 14, but the number of the second objects 120 is not limited to this example. Moreover, the second object 120a and the second object 120b belong to different frequency bands in FIG. 14, but may belong to the same or overlapping frequency bands.

Meanwhile, the program that is executed by each of the devices in the embodiment and the modifications as described above is provided by being incorporated in a ROM, a storage unit, or the like in advance. The program executed by each of the devices of the embodiment and the modifications as described above may be provided by being recorded in a computer readable recording medium, such as a compact disk (CD)-ROM, a flexible disk (FD), or a digital versatile disk (DVD), in a computer-installable or computer-executable file format.

Furthermore, the program executed by each of the devices of the embodiment and the modifications as described above may be stored in a computer that is connected to a network, such as the Internet, and may be provided by download via the network. Moreover, the program executed by each of the devices of the embodiment and modifications as described above may be provided or distributed via a network, such as the Internet.

According to an embodiment, it is possible to measure a biological reaction of a subject against a visual stimulus with high accuracy.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A biological signal measurement system comprising:
a providing unit configured to provide a first visual stimulus and a second visual stimulus to a subject, the first visual stimulus including a first object visually changing at a predetermined frequency, the second visual stimulus including a second object;
a detection unit configured to detect a biological signal of the subject;
a frequency analysis unit configured to perform a frequency analysis on the biological signal detected by the detection unit and corresponding to the first visual stimulus, and derive a signal intensity of each frequency component;
a determination unit configured to determine a time interval in which the subject has viewed the first visual stimulus, based on a signal intensity of a frequency component corresponding to the frequency;
an extraction unit configured to extract the biological signal corresponding to the time interval determined by the determination unit, and corresponding to the second visual stimulus; and
an output unit configured to output the biological signal extracted by the extraction unit.

2. The biological signal measurement system according to claim 1, wherein the determination unit is configured to compare the signal intensity of the frequency component corresponding to the frequency and a threshold, and determine, as the time interval in which the subject has viewed the first visual stimulus, a time interval including at least a period in which the signal intensity is larger than the threshold.

3. The biological signal measurement system according to claim 1, wherein
the providing unit is configured to continuously provide the first visual stimulus and the second visual stimulus as a single trial, and
the frequency analysis unit is configured to perform the frequency analysis based on the biological signal detected in a provision time corresponding to the single trial.

4. The biological signal measurement system according to claim 3, wherein the frequency analysis unit is configured to perform the frequency analysis using the biological signal corresponding to at least two trials.

5. The biological signal measurement system according to claim 1, wherein the providing unit is configured to visually change the first object at a frequency in a range from 60 Hz to 200 Hz.

6. The biological signal measurement system according to claim 1, wherein the providing unit is configured to change one of luminance, color, and a pattern of the first object at the frequency.

7. The biological signal measurement system according to claim 1, wherein the frequency analysis unit is configured to perform, as the frequency analysis, short-time Fourier transform on the biological signal.

8. The biological signal measurement system according to claim 1, wherein the frequency analysis unit is configured to perform a process of eliminating noise before or after the frequency analysis.

9. The biological signal measurement system according to claim 1, wherein the detection unit is configured to detect the biological signal indicating brain neural activity of the subject.

10. The biological signal measurement system according to claim 1, wherein the output unit is configured to output a result of averaging the biological signal extracted by the extraction unit.
